(19) United States
(12) Patent Application Publication
(10) Pub. No.: US 2021/0346321 A1
(43) Pub. Date: Nov. 11, 2021

(54) PHARMACEUTICAL COMPOSITION CONTAINING ALKYL CARBAMOYL NAPHTHALENYLOXY OCTENOYL HYDROXYAMIDE PHOSPHATE, TARTRATE OR COMBINATION THEREOF, AND PREPARATION METHOD THEREFOR

(71) Applicant: CRYSTALGENOMICS, INC., Gyeonggi-do (KR)

(72) Inventors: Jae Pyoung CHO, Gyeonggi-do (KR); Joong Myung CHO, Seoul (KR)

(21) Appl. No.: 17/283,627

(22) PCT Filed: Oct. 28, 2019
(86) PCT No.: PCT/KR2019/014234

§ 371 Date: Apr. 8, 2021

(30) Foreign Application Priority Data
Oct. 29, 2018 (KR) 10-2018-0129576

(51) Int. Cl. A61K 31/165; A61K 9/20; A61K 9/48

(52) U.S. Cl. CPC A61K 31/165; A61K 9/2018; A61K 9/2013; A61K 9/485; A61K 9/2054; A61K 9/4858; A61K 9/4866; A61K 9/2009

(57) ABSTRACT

The present invention relates to a pharmaceutical composition containing an alkyl carbamoyl naphthalenyloxy octenoyl hydroxyamide phosphate compound (hereinafter referred to as CG200745PPA) or a derivative thereof, and can provide a pharmaceutical composition in a tablet, granule, powder, capsule, dry syrup or injection form. By providing the composition in various forms, a suitable form can be selected and easily applied when oral administration is difficult.

### TECHNICAL FIELD

The present invention relates to a pharmaceutical composition comprising an alkylcarbamoyl naphthalenyloxy octenoyl hydroxyamide phosphate compound, an alkylcarbamoyl naphthalenyloxy octenoyl hydroxyamide tartrate compound or a combination thereof.

In addition, the present invention relates to a method for preparing the pharmaceutical composition.

### BACKGROUND ART

A histone is a basic protein that binds to DNA in the eukaryotic cell nucleus, and reversible histone acetylation occurs on the amino group of a specific lysine residue in each molecule of the histone. The histone acetylation reaction is related to the formation of high-order structure of chromatin or the cell-division cycle, so it is involved in the regulation of expression of gene information, and stably regulated by histone acetyltransferases (HATs) and histone deacetylases (HDACs). It is known that these enzymes neutralize the positive charge of lysine residues (4 residues in the case of H4) present at the amino terminal of the histone by acetylation to induce transcriptional activity or release electric charge again by deacetylation to inhibit transcription, thereby inducing an equilibrium of the level of histone acetylation to regulate gene expression at the level of transcription.

HDAC has recently been found to play a role in promoting cell proliferation as it is highly expressed in poor environmental conditions such as hypoxia, low glucose and cell carcinogenesis to inhibit the expression of cell proliferation. Therefore, it is recognized as an important factor in regulating cell carcinogenesis and differentiation. That is, while high acetylation of chromatin inhibits cell proliferation and promotes differentiation, HDAC plays a crucial role in inducing cell proliferation through deacetylation of histones. This is supported by the results of inhibition of cell proliferation or angiogenesis by treatment with HDAC inhibitors. There is a need for the development of HDAC inhibitors having higher selectivity and excellent medicinal efficacy. Accordingly, the present inventors confirmed the possibility of alkylcarbamoyl naphthalenyloxy octenoyl hydroxyamide as an HDAC inhibitor, and thus have been conducting research on this.

However, since the alkylcarbamoyl naphthalenyloxy octenoyl hydroxyamide has a property of absorbing moisture in the atmosphere, there may occur a problem that is vulnerable to physicochemical stability. A number of purification operations for removal of related substances generated by moisture absorption can increase the production cost, and high hygroscopicity makes difficult to maintain the solid state, causing difficulties in mass-producing solid preparations. There is a disadvantage in that a separate freezing storage device or means such as packaging is required.

Therefore, there is a need for a study on a pharmaceutical composition as a stabilized HDAC inhibitor, which has rapid expression of effects and is convenient to manufacture.

### DETAILED DESCRIPTION OF THE INVENTION

### Technical Problem

The present invention provides a pharmaceutical composition in the form of a tablet, granule, powder, capsule, dry syrup or injection comprising an alkylcarbamoyl naphthalenyloxy octenoyl hydroxyamide hydrochloride compound, an alkyl carbamoyl naphthalenyloxy octenoyl hydroxyamide tartrate compound or a combination thereof.

In addition, it provides a method for preparing the pharmaceutical composition.

### Solution to Problem

In order to solve the above problems, the present invention provides a pharmaceutical composition in the form of a tablet, granule, powder, capsule, dry syrup or injection comprising a compound of formula 1 (alkylcarbamoyl naphthalenyloxy octenoyl hydroxyamide phosphate), a compound of formula 2 (alkyl carbamoyl naphthalenyloxy octenoyl hydroxyamide tartrate) or a combination thereof.

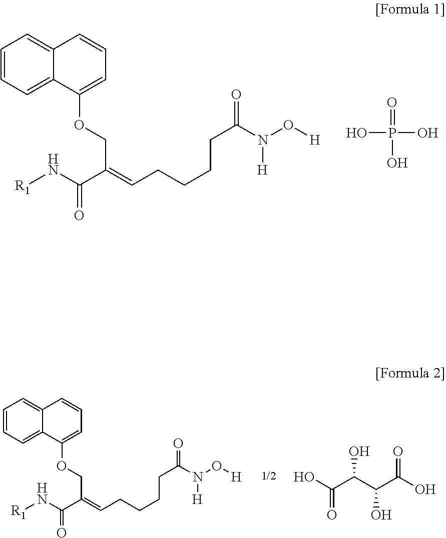

In the formula 1 or 2,

R₁ is C₁₋₃ alkyl unsubstituted or substituted with one or more substituents selected from the group consisting of halophenyl, C₁₋₃ alkoxy, C₁₋₃ alkoxy C₁₋₃ alkyl, cyclohexanyl, furanyl, thiophenyl, imidazole, imidazolidyl C₁₋₃ alkyl, C₁₋₃ alkylamino, di-C₁₋₃ alkylamino, hydroxyphenyl, tetrahydrofuranyl, cyclohexyl, cyclohexenyl, oxopyrrolidinyl, C₁₋₃ alkoxyphenyl, di-C₁₋₃ alkylaminophenyl, C₁₋₃ alkylpyrrolidinyl and trifluoromethoxyphenyl; pyrrolidine unsubstituted or substituted with C₃₋₈ cycloalkyl, C₃₋₈ cycloalkyl C₁₋₃ alkyl, benzyl, C₁₋₃ alkyl or C₃₋₈ cycloalkylcarbonyl; piperidine substituted with C₁₋₃ alkyl or C₃₋₈ cycloalkyl; furan; or C₃₋₈ cycloalkyl,

with proviso that unsubstituted C₁₋₂ alkyl and C₁₋₂ alkyl substituted with C₁₋₂ alkylpyrrolidinyl are excluded.

According to an embodiment, the coating layer may be present in an amount of 1 to 10% by weight based on 100 parts by weight of the tablet or capsule.

According to an embodiment, the composition may be a pharmaceutical composition for injection having a liquid or lyophilized form.

According to an embodiment, it may be provided a pharmaceutical composition for an anticancer agent comprising a compound of formula 1, a compound of formula 2 or a combination thereof.

According to another aspect, the present invention provides a method for preparing a pharmaceutical composition in the form of a tablet, granule, powder, capsule, dry syrup or injection, the method comprising:

1) adding an organic solvent to an alkylcarbamoyl naphthalenyloxy octenoyl hydroxyamide compound or a derivative thereof to extract a free base, and

2) adding phosphoric acid or tartaric acid to the solution of the free base.

According to an embodiment, the method may further comprise additionally adding a solvent having a lower solubility than the organic solvent of step 1).

According to an embodiment, the organic solvent of step 1) may comprise one or more selected from the group consisting of methanol, ethanol, propanol, tetrahydrofuran, chloroform, N,N-dimethylformamide (DMF), dimethyl sulfoxide (DMSO), acetonitrile and ethyl acetate.

According to an embodiment, the solvent having a lower solubility than the organic solvent of step 1) may comprise one or more selected from the group consisting of alcohols including methanol, ethanol and propanol, tetrahydrofuran, acetonitrile and acetone.

According to an embodiment, the method may further comprise sterilizing by a high-temperature decompression sterilization method or aseptic filtration method.

Other specifics of the embodiments of the present invention are included in the detailed description below.

### Effect of the Invention

According to the alkylcarbamoyl naphthalenyloxy octenoyl hydroxyamide phosphate compound, the alkyl carbamoyl naphthalenyloxy octenoyl hydroxyamide tartrate compound, or combinations thereof of the present invention, it is possible to improve stability against moisture while maintaining properties such as medicinal efficacy and effective amount. In addition, by improving hygroscopicity, production and commercialization processes of the preparations can be simplified. In particular, by maintaining stability during the manufacturing process to prevent the production of related substances, it is possible to provide a compound and pharmaceutical composition having safety and easy of manufacture.

### BRIEF DESCRIPTION OF DRAWINGS

FIG. 1 is a graph showing the dissolution pattern of tablets comprising alkylcarbamoyl naphthalenyloxy octenoyl hydroxyamide phosphate.

FIG. 2 is a graph showing the dissolution pattern of capsules comprising alkylcarbamoyl naphthalenyloxy octenoyl hydroxyamide phosphate.

FIG. 3 is a graph showing the results of measuring the amount of related substances produced in Example 12.

FIG. 4 is a graph showing a change in moisture content.

FIG. 5 is a graph comparing the amount of related substances produced in Examples and Comparative Examples.

FIG. 6 is a graph showing the amount of related substances produced in Example 13 according to storage temperature.

FIG. 7 is a graph showing the amount of related substances produced in Example 14 according to storage temperature.

FIG. 8 is a photograph showing appearance of the composition for injection having a lyophilized form comprising alkylcarbamoyl naphthalenyloxy octenoyl hydroxyamide phosphate.

### BEST MODE FOR CARRYING OUT THE INVENTION

Since various modifications and variations can be made in the present invention, particular embodiments are illustrated in the drawings and will be described in detail in the detailed description. It should be understood, however, that the invention is not intended to be limited to the particular embodiments, but includes all modifications, equivalents, and alternatives falling within the spirit and scope of the invention. In the following description of the present invention, detailed description of known functions will be omitted if it is determined that it may obscure the gist of the present invention.

The term “pharmaceutical composition”, as used herein may be described interchangeably with “pharmacological composition” and “pharmaceutically acceptable composition” and refers to any composition which can be a relatively non-toxic to a subject to be administered and have harmless effective action. In addition, it may refer to any organic or inorganic compound formulation in that side effects resulting from the composition do not impair the efficacy of the drug, and that does not cause serious irritation to a subject to be administered by the compound and does not impair the biological activities and properties of the compound.

As used herein, the term ‘subject to be administered’ may be used interchangeably with ‘subject to be applied’, ‘individual to be administered’ and ‘organism to be administered’, and may refer to any animals including humans in need of HDAC inhibition.

Hereinafter, the pharmaceutical composition comprising an alkylcarbamoyl naphthalenyloxy octenoyl hydroxyamide phosphate compound, an alkylcarbamoyl naphthalenyloxy octenoyl hydroxyamide tartrate compound or a combination thereof according to the embodiment of the present invention will be described in more detail.

The possibility of alkylcarbamoyl naphthalenyloxy octenoyl hydroxyamide or a derivative thereof as a histone deacetylase (HDAC) inhibitor has been confirmed (Korean Patent No. 0814092). In order to improve stability against moisture while maintaining properties such as medicinal efficacy and effective amount of alkylcarbamoyl naphthalenyloxy octenoyl hydroxyamide or its derivatives, the present inventors have studied the most physicochemically ideal and stable form of the salt among various types of acceptable salts.

In particular, the present invention provides a pharmaceutical composition comprising a compound of formula 1 (alkylcarbamoyl naphthalenyloxy octenoyl hydroxyamide phosphate), a compound of formula 2 (alkyl carbamoyl naphthalenyloxy octenoyl hydroxyamide tartrate) or a combination thereof.

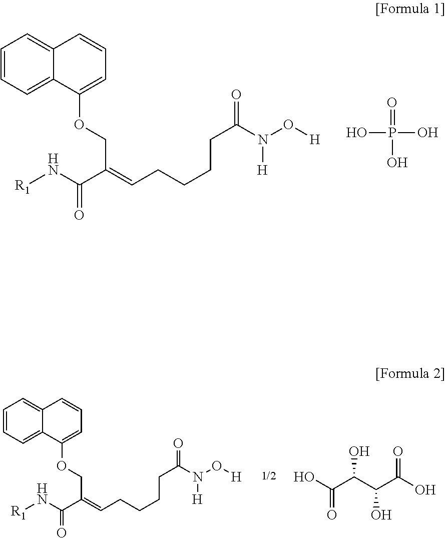

In the formula 1 or 2,

R₁ is C₁₋₃ alkyl unsubstituted or substituted with one or more substituents selected from the group consisting of halophenyl, C₁₋₃ alkoxy, C₁₋₃ alkoxy C₁₋₃ alkyl, cyclohexanyl, furanyl, thiophenyl, imidazole, imidazolidyl C₁₋₃ alkyl, C₁₋₃ alkylamino, di-C₁₋₃ alkylamino, hydroxyphenyl, tetrahydrofuranyl, cyclohexyl, cyclohexenyl, oxopyrrolidinyl, C₁₋₃ alkoxyphenyl, di-C₁₋₃ alkylaminophenyl, C₁₋₃ alkylpyrrolidinyl and trifluoromethoxyphenyl; pyrrolidine unsubstituted or substituted with C₃₋₈ cycloalkyl, C₃₋₈ cycloalkyl C₁₋₃ alkyl, benzyl, C₁₋₃ alkyl or C₃₋₈ cycloalkylcarbonyl; piperidine substituted with C₁₋₃ alkyl or C₃₋₈ cycloalkyl; furan; or C₃₋₈ cycloalkyl, with proviso that unsubstituted C₁₋₂ alkyl and C₁₋₂ alkyl substituted with C₁₋₂ alkylpyrrolidinyl are excluded.

In addition, according to a specific embodiment, R₁ may be N,N-dimethylpropylamine.

The present invention may provide an alkylcarbamoyl naphthalenyloxy octenoyl hydroxyamide compound containing phosphate or tartrate, such as a compound of formula 1 or a compound of formula 2. Preferred compounds as alkylcarbamoyl naphthalenyloxy octenoyl hydroxyamide derivatives may be selected from the group consisting of the following compounds:
1) (E)-N1-(3-(1H-imidazol-1-yl)propyl)-N8-hydroxy-2-((naphthalen-1-yloxy)methyl)octenediamide,
2) (E)-N8-hydroxy-N1-(4-hydroxyphenethyl)-2-((naphthalen-1-yloxy)methyl)-2-octenediamide,
3) (E)-N1-(3-(dimethylamino)-2,2-dimethylpropyl)-N8-hydroxy-2-((naphthalen-1-yloxy)methyl)octenediamide,
4) (E)-N1-(2-(diisopropylamino)ethyl)-N8-hydroxy-2-((naphthalen-1-yloxy)methyl)octenediamide,
5) (E)-N8-hydroxy-N1-(1-methoxypropan-2-yl)-2-((naphthalen-1-yloxy)methyl)-2-octenediamide,
6) (E)-N8-hydroxy-N1-(4-methoxybenzyl)-2-((naphthalen-1-yloxy)methyl)-2-octenediamide,
7) (E)-N1-(4-fluorophenethyl)-N8-hydroxy-2-((naphthalen-1-yloxy)methyl)-2-octenediamide,
8) (E)-N8-hydroxy-2-((naphthalen-1-yloxy)methyl)-N1-(tetrahydrofuran-2-yl)methyl)-2-octenediamide,
9) (E)-N1-(2-cyclohexenylethyl)-N8-hydroxy-2-((naphthalen-1-yloxy)methyl)-2-octenediamide,
10) (E)-N8-hydroxy-2-((naphthalen-1-yloxy)methyl)-N1-(3-(2-oxopyrrolidin-1-yl)propyl)-2-octenediamide,
11) (E)-N1-(furan-2-ylmethyl)-N8-hydroxy-2-((naphthalen-1-yloxy)methyl)-2-octenediamide,
12) (E)-N1-(4-(dimethylamino)benzyl)-N8-hydroxy-2-((naphthalen-1-yloxy)methyl)-2-octenediamide,
13) (E)-N8-hydroxy-N1-(2-methoxyethyl)-2-((naphthalen-1-yloxy)methyl)-2-octenediamide,
14) (E)-N1-cyclohexyl-N8-hydroxy-2-((naphthalen-1-yloxy)methyl)-2-octenediamide,
15) (E)-N8-hydroxy-2-((naphthalen-1-yloxy)methyl)-N1-(thiophen-2-ylmethyl)-2-octenediamide,
16) (E)-N8-hydroxy-N1-(4-methoxyphenethyl)-2-((naphthalen-1-yloxy)methyl)-2-octenediamide,
17) (E)-N8-hydroxy-2-((naphthalen-1-yloxy)methyl)-N1-(4-(trifluoromethoxy)benzyl)-2-octenediamide,
18) (E)-N1-(1-(cyclohexylmethyl)pyrrolidin-3-yl)-N8-hydroxy-2-((naphthalen-1-yloxy)methyl)-2-octenediamide,
19) (E)-N1-(1-cyclopentylpiperidin-4-yl)-N8-hydroxy-2-((naphthalen-1-yloxy)methyl)-2-octenediamide,
20) (E)-N1-(1-benzylpyrrolidin-3-yl)-N8-hydroxy-2-((naphthalen-1-yloxy)methyl)-2-octenediamide,
21) (E)-N8-hydroxy-N1-(1-isopropylpyrrolidin-3-yl)-2-((naphthalen-1-yloxy)methyl)-2-octenediamide,
22) (E)-N1-(1-(cyclohexanecarbonyl)pyrrolidin-3-yl)-N8-hydroxy-2-((naphthalen-1-yloxy)methyl)-2-octenediamide,
23) (E)-3-(8-(hydroxyamino)-2-((naphthalen-1-yloxy)methyl)-8-oxo-2-octenamido)pyrrolidine-1-carboxylic acid t-butyl ester,
24) (E)-N8-hydroxy-2-((naphthalen-1-yloxy)methyl)-N1-(pyrrolidin-3-yl)2-octenediamide,
25) (E)-N1-(1-cyclohexylpyrrolidin-3-yl)-N8-hydroxy-2-((naphthalen-2-yloxy)methyl)-2-octenediamide,
26) (E)-N1-(1-cyclopropylpyrrolidin-3-yl)-N8-hydroxy-2-((naphthalen-1-yloxy)methyl)-2-octenediamide,
27) (E)-N1-(1-cyclopropylpiperidin-4-yl)-N8-hydroxy-2-((naphthalen-1-yloxy)methyl)-2-octenediamide,
28) (E)-N1-(1-ethylpiperidin-4-yl)-N8-hydroxy-2-((naphthalen-1-yloxy)methyl)-2-octenediamide,
29) (E)-N1-(1-ethylpyrrolidin-3-yl)-N8-hydroxy-2-((naphthalen-1-yloxy)methyl)-2-octenediamide,
30) (E)-N8-hydroxy-N1-(2-(1-methylpyrrolidin-2-yl)ethyl)-2-((naphthalen-1-yloxy)methyl)-2-octenediamide,
31) (E)-N8-hydroxy-N1-(1-isopropylpiperidin-4-yl)-2-((naphthalen-1-yloxy)methyl)-2-octenediamide, and
32) (E)-N1-(3-(dimethylamino)propyl)-N8-hydroxy-2-((naphthalen-1-yloxy)methyl)-2-octenediamide.

According to another embodiment of the present invention, there is provided a method for preparing a pharmaceutical composition comprising a compound of formula 1, a compound of formula 2, or a combination thereof, comprising:

1) adding an organic solvent to an alkylcarbamoyl naphthalenyloxy octenoyl hydroxyamide compound or a derivative thereof to extract a free base, and

2) adding phosphoric acid or tartaric acid to the solution of the free base.

According to an embodiment, the alkylcarbamoyl naphthalenyloxy octenoyl hydroxyamide or a derivative thereof may be in the form of a solid, gel or solution wherein the form of solution may refer to a state completely dissolved in an organic solvent or a suspension state.

According to an embodiment, the organic solvent may comprise one or more selected from the group consisting of methanol, ethanol, propanol, tetrahydrofuran, chloroform, N,N-dimethylformamide (DMF), dimethyl sulfoxide (DMSO), acetonitrile and ethyl acetate. For example, it may comprise one or more selected from the group consisting of methanol, ethanol, propanol, tetrahydrofuran, chloroform, N,N-dimethylformamide (DMF) and dimethyl sulfoxide (DMSO), which have relatively high solubility.

According to an embodiment, the method may further comprise additionally adding a solvent having a lower solubility than the organic solvent of step 1). For example, the solvent having a lower solubility than the organic solvent of step 1) may comprise one or more selected from the group consisting of alcohols including methanol, ethanol and propanol, tetrahydrofuran, acetonitrile and acetone. For example, after adding an organic solvent to alkylcarbamoyl naphthalenyloxy octenoyl hydroxyamide or a derivative thereof, the precipitation, that is, the formation of a salt is observed, and if necessary, a solvent with a lower solubility than the added organic solvent may be additionally added to observe the precipitation. The addition of the solvent having a lower solubility may be repeated 2 to 5 times, for example, 2 times to obtain a salt.

The pharmaceutical composition of the present invention may be provided in the form of a tablet, granule, powder, capsule, dry syrup or injection. Specifically, it may be provided in any convenient form, such as in the form of tablets, pellets, granules, capsules, suspensions, emulsions or powders, which are suitable for reconstitution with water or other suitable liquid medium. In addition, it may be provided in the form of oral administration or injection.

The pharmaceutical composition for oral administration may comprise at least one diluent selected from the group consisting of microcrystalline cellulose, mannitol, lactose and lactose, at least one lubricant selected from the group consisting of talc, magnesium stearate, sodium stearyl fumarate and glyceryl behenate, and at least one binder selected from the group consisting of polyvinylpyrrolidone, hydroxypropyl methylcellulose and hydroxypropyl cellulose. In addition, for example, the coating layer may be included in an amount of 1 to 10% by weight based on 100% by weight of a tablet or capsule. Specifically, for example, the coating layer may include a water-soluble coating base material, and a commonly used coating base material may be used. More specifically, for example, it includes a coating base material including polyvinyl alcohol derivatives, methacrylic acid derivatives and polyacrylic acid derivatives, and for example, one or two or more selected from the group consisting of Opadry®, Kollicoat®, and hydroxypropyl methylcellulose (IPMC) may be used, for example polyvinyl alcohol containing Opadry® having a relatively excellent effect of blocking moisture and light may be used.

When preparing a granule composition for oral administration, it is preferable not to use purified water as a binding solvent in consideration of the property of unstable moisture stability. Ethanol may be used that can be easily removed during the manufacturing process. Magnesium stearate, which is a lubricant used in a conventional composition for oral administration, may not be suitable for mixing with the alkylcarbamoyl naphthalenyloxy octenoyl hydroxyamide according to the present invention, so alternatives can be used.

According to an embodiment, in addition to the additives as described above, a pharmaceutical excipient having excellent compatibility with the compound can be added.

The injection composition can be provided in a liquid form because the alkylcarbamoyl naphthalenyloxy octenoyl hydroxyamide component is a water-soluble material that is well soluble in water. The use of solubilizing agents and other additives generally used to improve solubility of poorly soluble substances are not essential. It is preferable to use a minimum amount of additive as it may have unstable compatibility with the additives. More specifically, it can be prepared by dissolving in nitrogen-purged water for injection and then lyophilizing.

According to an embodiment, the method may further comprise sterilizing. The sterilization method may include dry heat sterilization, pressurized or reduced pressure sterilization, filter sterilization, gas sterilization, radiation sterilization, and the like. In the filter sterilization method, a nitrocellulose membrane filter may be used, for example, and the filter having 0.45 μm or 0.2 μm pore size may be used. In the present invention, the method may further comprise sterilizing by a high-temperature decompression sterilization method or aseptic filtration method.

According to another embodiment of the present invention, it is possible to provide a pharmaceutical composition for an anticancer agent containing the compound of formula 1, the compound of formula 2, or a combination thereof as described above as an effective ingredient.

Hereinafter, embodiments of the present invention will be described in detail so that those skilled in the art can easily carry out the present invention. The present invention may, however, be embodied in many different forms and should not be construed as limited to the embodiments set forth herein.

### Examples 1 to 4

In order to prepare a tablet composition having convenience of dosing and easy of manufacture, a tablet composition was prepared using alkylcarbamoyl naphthalenyloxy octenoyl hydroxyamide phosphate and additives having established stability in the compositions as shown in Table 1, based on the results of compatibility studies.

(E)-N1-(3-(dimethylamino)propyl)-N8-hydroxy-2-((naphthalen-1-yloxy)methyl)-2-octenediamide phosphate compound (CG200745PPA) was used as alkylcarbamoyl naphthalenyloxy octenoyl hydroxyamide phosphate. The weight in the table below is in mg.

Owing to the nature of poor flowability of CG200745PPA, a tablet composition was prepared by a granulation method using ethanol as a binding solvent, instead of a direct compression method. In addition, due to the characteristics of CG200745PPA raw materials, it can be difficult to separate the tablet and the punch when subjected to the tableting pressure in the tableting process. Therefore, the ratio of the excipient added was increased, thereby minimizing the influence of appearance of the main component.

In addition, in Table 2, according to the degree to which the composition is jammed in the machine when punching the tablet, the mold release is expressed as good and poor. According to the degree of sticking to the machine surface, the sticking is expressed as ∘: non-sticking, X: sticking and Δ: good. According to the cracking of the layer of the tablet after tableting, the capping is expressed as ∘: no capping and X: occurrence of capping. The mold release and sticking were evaluated by visual observation, and the degree of capping was evaluated by separating the tablet by applying a physical force to the tablet by hand.

TABLE 1

| | Manufacturing | | | | | |
|---|---|---|---|---|---|---|
| | Process | Raw material | Example 1 | Example 2 | Example 3 | Example 4 |
| 1 | Granulation | CG200745PPA | 125 | 125 | 125 | 125 |
| 2 | | Mannitol | 200 | 200 | 200 | 200 |
| 3 | | Microcrystalline | 200 | 200 | 200 | 200 |
| | | cellulose | | | | |
| 4 | | Silicon dioxide | 5 | 5 | 5 | 5 |
| 6 | | Magnesium | 10 | 10 | 10 | 10 |
| | | aluminometasilicate | | | | |
| 7 | | Hydroxypropyl | 15 | 15 | 15 | 15 |
| | | cellulose | | | | |
| 8 | Post mixing | Glyceryl behenate | 10 | 10 | 30 | 50 |
| 9 | | Talc | 10 | — | 10 | 10 |
| 10 | | Sodium stearyl | — | 10 | — | — |
| | | fumarate | | | | |

| | | | | |
|---|---|---|---|---|
| Total weight | 575 | 575 | 595 | 615 |

TABLE 2

| Evaluation of | | | | |
|---|---|---|---|---|
| tableting | | | | |
| properties | Example 1 | Example 2 | Example 3 | Example 4 |
| Sticking | ◯ | ◯ | ◯ | ◯ |
| Mold release | Poor | Poor | Poor | Poor |
| Capping | X | X | X | X |

As a result of Examples 1 to 4, it was found that even when the ratio of the main ingredients to the excipient was 2:8, the tablet was jammed in the punch, making it difficult to be ejected, and a sticking phenomenon occurred in which the mixture adhered to the punch surface. These were not significantly improved even by changing the type of the lubricant or increasing the amount of the lubricant.

### Examples 5 to 7

In order to improve the sticking phenomenon, tablets were manufactured by increasing the amount of excipients and decreasing the portion of the main ingredients as shown in Table 3. In addition, Table 4 shows the results of tableting evaluation.

TABLE 3

| | Manufacturing | | | | | |
|---|---|---|---|---|---|---|
| | Process | Raw material | Example 5 | Example 6 | Example 7 | Example 8 |
| 1 | Granulation | CG200745PPA | 125 | 125 | 125 | 125 |
| 2 | | Mannitol | 220 | 250 | 250 | 280 |
| 3 | | Microcrystalline | 200 | 220 | 220 | 220 |
| | | cellulose | | | | |
| 4 | | Silicon dioxide | 5 | 5 | 5 | 5 |
| 7 | | Hydroxypropyl | 30 | 30 | 30 | 30 |
| | | cellulose | | | | |
| 8 | Post mixing | Glyceryl behenate | 30 | 30 | 50 | 50 |
| 9 | | Talc | 10 | 10 | 10 | 10 |

| | | | | |
|---|---|---|---|---|
| Total weight | 620 | 670 | 690 | 720 |

TABLE 4

| Evaluation of | | | | |
|---|---|---|---|---|
| tableting | | | | |
| properties | Example 5 | Example 6 | Example 7 | Example 8 |
| Sticking | ◯ | ◯ | X | X |
| Mold release | Poor | Poor | Good | Good |
| Capping | X | X | X | X |

### Examples 9 and 10

CG200745PPA can be applied as an anticancer agent. Most of the subjects to be administered are patients undergoing chemotherapy. Therefore, if increasing in tablet size, it is not only very difficult to take, but also there is a fear of rejection in taking tablets. Accordingly, in the present invention, it was intended to improve it. A capsule formulation composition was prepared that has less physical influence on the manufacture and a simple process such as a tableting process. As shown in the composition of Table 5, for convenience of dosing, the minimum amount of excipients that can be manufactured was used, and for simplification of the process, the direct mixing was used. In addition, the results of evaluating the flowability according to the angle of repose measurement criteria as shown in Table 6 (General Chapters; 1174, USP) using an angle of repose tester and the degree of sticking are shown in Table 7.

EMBO CAPS (hard capsule, manufactured by Suheung) was used for capsule #0 and capsule #1.

TABLE 5

| | Manufacturing | | | |
|---|---|---|---|---|
| | Process | Raw material | Example 9 | Example 10 |
| 1 | Direct mixing | CG200745PPA | 125 | 125 |
| 2 | | Mannitol | 80 | 90 |
| 3 | | Microcrystalline | 70 | 80 |
| | | cellulose | | |
| 6 | | Magnesium | 5 | 10 |
| | | aluminometasilicate | | |
| 9 | | talc | 10 | 10 |
| 10 | | Sodium stearyl | 5 | 10 |
| | | fumarate | | |

| | | |
|---|---|---|
| Total weight | 295 | 325 |

TABLE 6
Angles of Repose

| Flow Property | Angle of Repose |
|---|---|
| Excellent | 25-30 |
| Good | 31-35 |
| Fair-aid not needed | 36-40 |
| Passable-may hang up | 41-45 |
| Poor-must agitate, vibrate | 46-55 |
| Very poor | 56-65 |
| Very, very poor | >66 |

TABLE 7

| Evaluation of | | Angle of | | |
|---|---|---|---|---|
| composition | Capsule # | repose | Sticking | Mass deviation |
| Example 9 | 1 | 46 | Δ | Mean 9% |
| Example 10 | 0 | 47 | X | Mean 7% |

### Examples 11 and 12

In order to improve flowability, a mixture was prepared in the form of granules, and a composition capable of filling in capsule #1 was prepared according to Table 8. In addition, the flowability, degree of sticking and mass deviation are shown in Table 9.

TABLE 8

| | Manufacturing | | | |
|---|---|---|---|---|
| | Process | Raw material | Example 11 | Example 12 |
| 1 | Granulation | CG200745PPA | 125 | 125 |
| 2 | | Mannitol | 75 | 60 |
| 3 | | Microcrystalline | 70 | 60 |
| | | cellulose | | |
| 6 | | Hydroxypropyl | 10 | 8 |
| | | cellulose | | |
| 9 | | Talc | 10 | 7 |

| | | |
|---|---|---|
| Total weight | 290 | 260 |

TABLE 9

| Evaluation of | | Angle of | | |
|---|---|---|---|---|
| composition | Capsule # | repose | Sticking | Mass deviation |
| Example 11 | 1 | 29 | X | 3% or less |
| Example 12 | 1 | 31 | X | 3% or less |

The capsules filled with granules showed good flowability and little mass deviation. A total weight of 290 mg was considered to be problematic when filling into Capsule #1 due to the large volume characteristics of the mixture. Accordingly, a composition with a total weight of mixture of 260 mg was selected.

### Experimental Example 1: Evaluation of Dissolution of Tablet

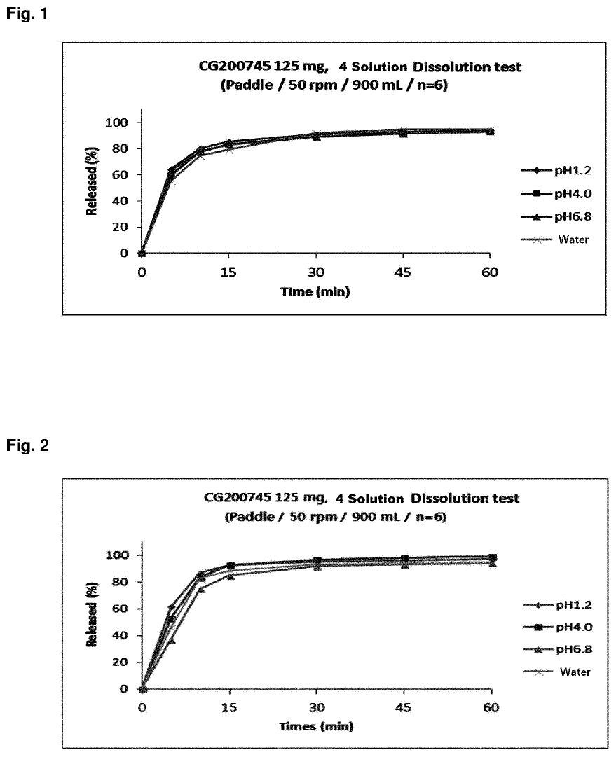

In accordance with Korean Pharmacopoeia dissolution test method 2 (paddle method, apparatus 2), the formulation of Example 8 was subjected to an in vitro dissolution test. The dissolution tests were carried out in all 4 solutions, and the results are shown in FIG. 1 and Table 10.

TABLE 10

| Solution | Min | 0 | 5 | 10 | 15 | 30 | 45 | 60 |
|---|---|---|---|---|---|---|---|---|
| pH 1.2 | Mean | 0 | 64.3 | 80.4 | 85.5 | 90.9 | 92.9 | 94.1 |
| | SD | 0 | 3.1 | 3.3 | 3 | 2.3 | 2 | 1.8 |
| pH 4.0 | Mean | 0 | 59.7 | 77.8 | 83.3 | 89.3 | 91.8 | 93 |
| | SD | 0 | 3.7 | 2.4 | 2.1 | 1.2 | 1.1 | 0.8 |
| pH 6.8 | Mean | 0 | 63.3 | 77.6 | 83.4 | 89 | 91.5 | 93.7 |
| | SD | 0 | 4.3 | 5 | 2.1 | 2 | 1.2 | 1.2 |
| water | Mean | 0 | 55.7 | 74.8 | 79.1 | 91.8 | 94.6 | 94.8 |
| | SD | 0 | 4.8 | 3.3 | 2.5 | 1.7 | 1.3 | 1.5 |

### Experimental Example 2: Evaluation of Dissolution of Capsule

In the same manner as in Experimental Example 1, in accordance with Korean Pharmacopoeia dissolution test method 2 (paddle method, apparatus 2), the formulation of Example 12 was subjected to an in vitro dissolution test. The dissolution tests were carried out in all 4 solutions, and the results are shown in FIG. 2 and Table 11.

TABLE 11

| Solution | min | 0 | 5 | 10 | 15 | 30 | 45 | 60 |
|---|---|---|---|---|---|---|---|---|
| pH 1.2 | Mean | 0 | 61.7 | 86.7 | 92.4 | 95.3 | 96.2 | 97 |
| | SD | 0 | 13.6 | 9.1 | 4.7 | 4 | 3.1 | 3.3 |
| pH 4.0 | Mean | 0 | 53 | 83.5 | 92.6 | 96.9 | 98.3 | 99.2 |
| | SD | 0 | 7.7 | 8.4 | 4.6 | 3.3 | 2.3 | 2.1 |
| pH 6.8 | Mean | 0 | 37.1 | 74.8 | 84.8 | 91.7 | 93.2 | 94 |
| | SD | 0 | 6.1 | 6.6 | 2.3 | 1.9 | 1.7 | 1.3 |
| water | Mean | 0 | 46.2 | 82.8 | 88.7 | 93.1 | 94.1 | 94.9 |
| | SD | 0 | 7.4 | 6 | 3.8 | 2.7 | 2.1 | 1.7 |

### Experimental Example 3: Evaluation of Related Substances of Capsule Composition of Example 12

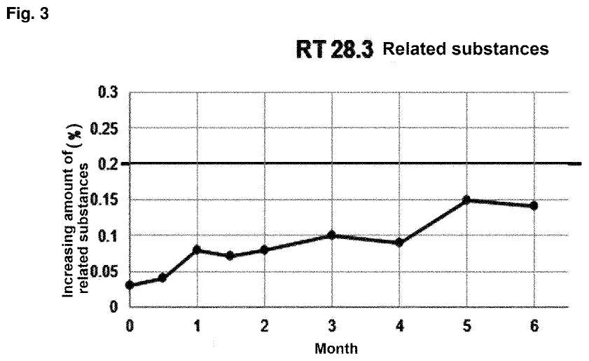

In order to evaluate the amount of change in the related substances of Example 12, the composition of Example 12 was placed in an accelerated condition chamber (45° C.±2, 75%±5), and the amount of related substance produced was measured for 6 months. For the measurement of the amount of related substances produced, the composition of Example 12 was pretreated and then tested according to the liquid chromatography method of the general test method of the Korean Pharmacopoeia to determine peak areas of the produced related substances (individual related substance 0.2% or less, total related substances 1.0%, measuring equipment: HPLC LC-2030C_Shimadzu). The results are shown in Table 12 and FIG. 3. In Table 12, the amount of related substances produced is expressed in %. In addition, FIG. 3 is a result of measuring the related substance at 28 minutes under the accelerated condition, and the reference value is 0.2%.

TABLE 12

| | RT(min) | | | | | | | | | | |
|---|---|---|---|---|---|---|---|---|---|---|---|
| Month | 4.7 | 6.8 | 7.8 | 8.0 | 9.4 | 9.8 | 10.4 | 15.6 | 16.7 | 28.3 | 42.0 |
| 0 | 0.05 | — | 0.02 | 0.07 | 0.03 | — | 0.03 | 0.03 | 0.06 | 0.03 | 0.09 |
| 0.5 | 0.05 | 0.01 | 0.02 | 0.06 | 0.04 | 0.03 | 0.03 | 0.03 | 0.04 | 0.04 | 0.08 |
| 1 | 0.08 | 0.04 | 0.04 | 0.06 | 0.03 | 0.02 | 0.03 | 0.04 | 0.05 | 0.08 | 0.08 |
| 1.5 | 0.05 | — | — | 0.08 | 0.03 | 0.03 | — | 0.04 | 0.07 | 0.07 | 0.07 |
| 2 | 0.06 | 0.04 | 0.03 | 0.05 | — | 0.03 | 0.02 | 0.03 | 0.05 | 0.08 | 0.07 |
| 3 | 0.05 | | 0.01 | 0.04 | 0.02 | 0.02 | 0.02 | 0.03 | 0.04 | 0.10 | 0.07 |
| 4 | 0.05 | 0.03 | 0.03 | 0.05 | | 0.02 | 0.03 | 0.04 | 0.05 | 0.09 | 0.06 |
| 5 | 0.05 | 0.05 | 0.03 | 0.02 | 0.03 | 0.03 | | 0.03 | 0.05 | 0.15 | 0.05 |
| 6 | 0.06 | | | | 0.04 | | 0.03 | 0.04 | 0.03 | 0.14 | 0.05 |

RT: Retention time

As shown in Table 12 and FIG. 3, the amounts of other related substances produced except for the peak of RT 28.3 mins were insignificant. Further, it was confirmed that the amount of the related substance for the peak of RT 28.3 meets the criteria for individual related substance for 6 months under the accelerated condition.

### Experimental Example 4: Evaluation of Stability

In order to evaluate the stability of the compound according to the present invention, an alkylcarbamoyl naphthalenyloxy octenoyl hydroxyamide compound was used in Comparative Example 1, an alkyl carbamoyl naphthalenyloxy octenoyl hydroxyamide tartrate was used in Example 13 and alkylcarbamoyl naphthalenyloxy octenoyl hydroxyamide phosphate was used in Example 14. As alkylcarbamoyl naphthalenyloxy octenoyl hydroxyamide, (E)-N1-(3-(dimethylamino)propyl)-N8-hydroxy-2-((naphthalen-1-yloxy)methyl)-2-octenediamide compound was used. Evaluation of stability was conducted by observing the change with storage time under room temperature (20-25° C., 50% or less), long-term (25±2° C. and 60±5% RH), accelerated (40±2° C. and 75±5% RH) and severe (60±2° C.) conditions.

### Experimental Example 4-1: Evaluation of Change in Moisture Content

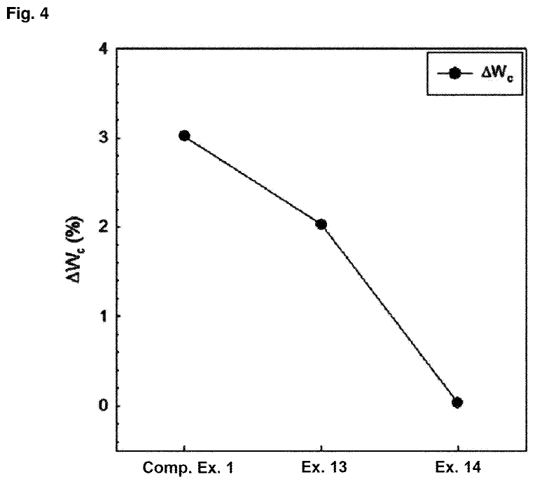

In order to evaluate the change in moisture content, the compounds according to Examples and Comparative Examples were stored in an open state at room temperature (20 to 25° C., 50% or less), and the change was measured by the difference between the initial moisture content and the moisture content after 3 days, and the results are shown in FIG. 4. Moisture content was measured according to the volumetric titration method among methods of determining the water content (Karl Fischer method) of the general test methods of the Korean Pharmacopoeia (measurement equipment: 701KF_Metrohm).

As shown in FIG. 4, in the case of Comparative Example 1, the moisture content was increased by about 3%, in the case of Example 13, the moisture content was increased by 2%, and in the case of Example 14, the change in the moisture content was very slight as about 0.01%.

### Experimental Example 4-2: Evaluation of Change in Appearance

In order to evaluate the change in appearance of the compounds according to Comparative Examples and Examples, each compound was stored in an open state according to the conditions shown in Table 13 below, and then the change in appearance was observed.

TABLE 13

| | Storage | Room | | | |
|---|---|---|---|---|---|
| Example | period | temperature | Long-term | Accelerated | Severe |
| Comp. | Initial | Foam | | | |
| Ex. 1 | 1 day | Gel-like | Gel-like | Gel-like | Gel-like |
| | 7 days | — | — | — | — |
| Ex. 13 | Initial | Powder | | | |
| | 1 day | Powder | Gel-like | Gel-like | Powder |
| | 7 days | Powder | — | — | Powder |
| | | (white) | | | Discoloration |
| | | | | | (pale yellow) |
| | | | | | (Particle size |
| | | | | | reduction) |
| Ex. 14 | Initial | Powder | | | |
| | 1 day | Powder | Powder | Powder | Powder |
| | 7 days | Powder | Powder | Powder | Powder |

As shown in Table 13, in the case of Comparative Example 1, as moisture was absorbed, the appearance was changed from a foam to a highly viscous liquid or gel after 1 day (24 hours) elapsed, and in the case of Example 14, it can be found that the appearance remained constant regardless of the condition.

### Experimental Example 4-3: Evaluation of Change in Content

In order to evaluate the change in the content of the compounds according to Examples, each compound was stored in an open state or in a polyethylene bottle packaging (silica gel added) to measure the change in content. For measurement of content, compounds of Comparative Examples and Examples were pretreated and then the solutions were tested according to the liquid chromatography method of the general test methods of the Korean Pharmacopoeia to determine the peak area of CG200745 (measurement equipment: HPLC LC-2030C_Shimadzu). The content standard is to contain CG200745 in an amount corresponding to 95.0-105.5% of the indicated amount according to the standards and test methods of the present applicant.

The results measured in the open state are shown in Table 14 below, and the results measured in the bottle packaging are shown in Table 15 below.

TABLE 14

| | Storage | Room | | | |
|---|---|---|---|---|---|
| Example | period | temperature | Long-term | Accelerated | Severe |
| Comp. | Initial | 98.9 | | | |
| Ex. 1 | 1 day | 101.0 | 99.9 | 92.3 | 101.1 |
| | 3 days | 101.9 | N.T. | N.T. | 97.2 |
| | 7 days | 100.1 | N.T. | N.T. | 89.1 |
| Ex. 13 | Initial | 99.5 | | | |
| | 1 day | 103.4 | 104.3 | 104.6 | 103.3 |
| | 3 days | 102.6 | 102.6 | 102.3 | 99.6 |
| | 7 days | 100.0 | 99.4 | 100.7 | 102.2 |

N.T: As the change in appearance was observed, the test was not performed.

TABLE 15

| | | Room | | | |
|---|---|---|---|---|---|
| Example | Storage period | temperature | Long-term | Accelerated | Severe |
| Comp. | Initial | 98.9 | | | |
| Ex. 1 | 1 day | 99.3 | 99.1 | 89.1 | 101.2 |
| | 3 days | 99.5 | 84.0 | 84.0 | N.T. |
| | 7 days | 99.4 | 78.5 | 78.5 | N.T. |
| Ex. 14 | Initial | 99.5 | | | |
| | 1 day | 98.6 | 103.5 | 101.2 | 101.6 |
| | 3 days | 98.8 | 99.9 | 100.1 | 100.4 |
| | 7 days | 99.3 | 100.9 | 101.1 | 101.4 |

N.T: As the change in appearance was observed, the test was not performed.

Summarizing the results of Experimental Example 4, it was found that in the case of Example 13, the content was maintained at a certain level under the room temperature condition, and in the case of Example 14, the content was kept constant regardless of packaging conditions, heating, and humidity.

### Experimental Example 5: Comparison of the Amount of Change in Related Substances

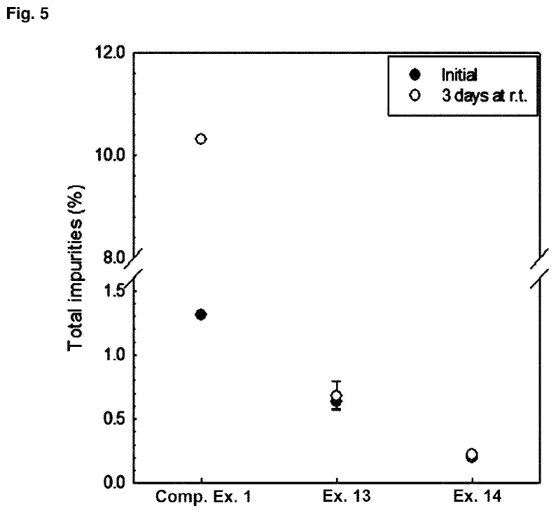

In order to evaluate the amount of change in related substances of the compounds according to Comparative Examples and Examples, each compound was stored in an open state at room temperature for 3 days, and then the amount of related substances produced was measured in the same manner as in Experimental Example 3 and the results are shown in FIG. 5. As shown in FIG. 5, the amount of related substances was increased by about 10% in Comparative Example 1, while the amount was increased by 0.05% in Example 13 and the amount was increased by 0.02% in Example 14, which were relatively insignificant.

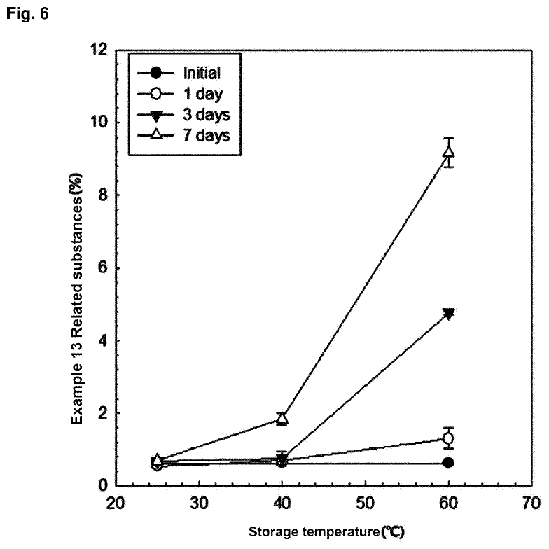

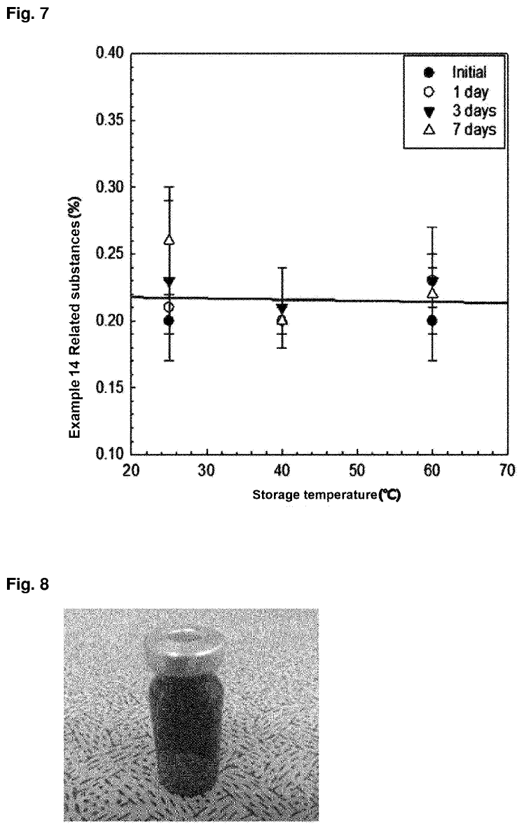

In addition, changes in the amount of related substances produced according to temperature and storage period are shown in FIGS. 6 and 7 for the compounds according to Examples 13 and 14. As shown in FIG. 6, the amount of related substances produced in Example 13 was maintained within a certain range at a relatively low temperature. As shown in FIG. 7, the amount of related substances produced in Example 14 was kept constant within a certain range of about 0.22% regardless of the storage temperature and period.

### Example 15

Since CG200745PPA is an anticancer drug that can be administered to cancer patients for the purpose of cancer treatment, various routes of administration should be considered according to the patient's condition. Thus, the route that can be administered to a subject having difficulties in oral administration includes intravenous or intramuscular injection. For this, there are injections in a liquid form or a lyophilized form that is diluted immediately before use. The composition as shown in Table 16 without using a solubilizing agent and other additives for improving stability in the dissolved state and compatibility with other additives, was dissolved in water for injection purged with nitrogen, and lyophilized. A photograph of the prepared composition is shown in FIG. 8.

TABLE 16

| | Raw material | Example 15 |
|---|---|---|
| 1 | CG200745PPA | 125 |
| 2 | Water for injection | To 5 mL |
| | (specific gravity 1.0 g/mL) | |
| 3 | Nitrogen | q.s. |

As can be seen from the above results, the CG200745PPA compound according to the present invention can improve the dissolution rate while maintaining properties such as medicinal efficacy and effective amount.

Therefore, it can be easily applied to a subject to be administered as an anticancer agent related to HDAC inhibition, for example, oral administration and injection.

The above descriptions are merely illustrative of the technical idea of the present invention, and those of ordinary skill in the technical field to which the present invention pertains can make various modifications and variations without departing from the essential characteristics of the present invention. In addition, the embodiments disclosed in the present invention are not intended to limit the technical idea of the present invention, but to explain the technical idea, and the scope of the technical idea of the present invention is not limited by these embodiments. The scope of protection of the present invention should be interpreted by the appended claims, and all technical ideas within the scope equivalent thereto should be interpreted as being included in the scope of the present invention.

What is claimed is:

1. A pharmaceutical composition comprising a compound of formula 1, a compound of formula 2 or a combination thereof in the form of a tablet, granule, powder, capsule, dry syrup or injection: 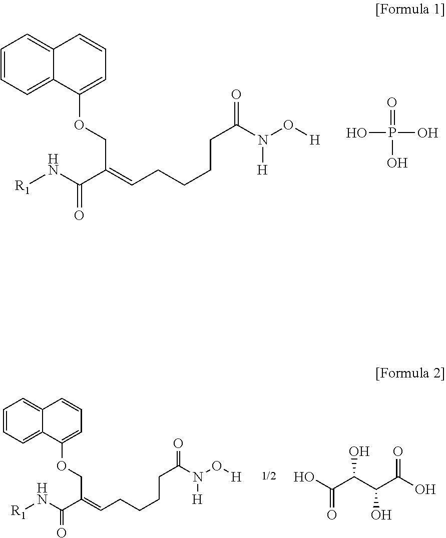
in the formula 1 or 2,
R₁ is C₁₋₃ alkyl unsubstituted or substituted with one or more substituents selected from the group consisting of halophenyl, C₁₋₃ alkoxy, C₁₋₃ alkoxy C₁₋₃ alkyl, cyclohexanyl, furanyl, thiophenyl, imidazole, imidazolidyl C₁₋₃ alkyl, C₁₋₃ alkylamino, di-C₁₋₃ alkylamino, hydroxyphenyl, tetrahydrofuranyl, cyclohexyl, cyclohexenyl, oxopyrrolidinyl, C₁₋₃ alkoxyphenyl, di-C₁₋₃ alkylaminophenyl, C₁₋₃ alkylpyrrolidinyl and trifluoromethoxyphenyl; pyrrolidine unsubstituted or substituted with C₃₋₈ cycloalkyl, C₃₋₈ cycloalkyl C₁₋₃ alkyl, benzyl, C₁₋₃ alkyl or C₃₋₈ cycloalkylcarbonyl; piperidine substituted with C₁₋₃ alkyl or C₃₋₈ cycloalkyl; furan; or C₃₋₈ cycloalkyl,
with proviso that unsubstituted C₁₋₂ alkyl and C₁₋₂ alkyl substituted with C₁₋₂ alkylpyrrolidinyl are excluded.

2. The pharmaceutical composition according to claim 1, wherein the alkylcarbamoyl naphthalenyloxy octenoyl hydroxyamide of formula 1 or 2 is selected from the group consisting of the following compounds:
1) (E)-N1-(3-(1H-imidazol-1-yl)propyl)-N8-hydroxy-2-((naphthalen-1-yloxy)methyl)octenediamide,
2) (E)-N8-hydroxy-N1-(4-hydroxyphenethyl)-2-((naphthalen-1-yloxy)methyl)-2-octenediamide,
3) (E)-N1-(3-(dimethylamino)-2,2-dimethylpropyl)-N8-hydroxy-2-((naphthalen-1-yloxy)methyl)octenediamide,
4) (E)-N1-(2-(diisopropylamino)ethyl)-N8-hydroxy-2-((naphthalen-1-yloxy)methyl)octenediamide,
5) (E)-N8-hydroxy-N1-(1-methoxypropan-2-yl)-2-((naphthalen-1-yloxy)methyl)-2-octenediamide,
6) (E)-N8-hydroxy-N1-(4-methoxybenzyl)-2-((naphthalen-1-yloxy)methyl)-2-octenediamide,
7) (E)-N1-(4-fluorophenethyl)-N8-hydroxy-2-((naphthalen-1-yloxy)methyl)-2-octenediamide,
8) (E)-N8-hydroxy-2-((naphthalen-1-yloxy)methyl)-N1-(tetrahydrofuran-2-yl)methyl)-2-octenediamide,
9) (E)-N1-(2-cyclohexenylethyl)-N8-hydroxy-2-((naphthalen-1-yloxy)methyl)-2-octenediamide,
10) (E)-N8-hydroxy-2-((naphthalen-1-yloxy)methyl)-N1-(3-(2-oxopyrrolidin-1-yl)propyl)-2-octenediamide,
11) (E)-N1-(furan-2-ylmethyl)-N8-hydroxy-2-((naphthalen-1-yloxy)methyl)-2-octenediamide,
12) (E)-N1-(4-(dimethylamino)benzyl)-N8-hydroxy-2-((naphthalen-1-yloxy)methyl)-2-octenediamide,
13) (E)-N8-hydroxy-N1-(2-methoxyethyl)-2-((naphthalen-1-yloxy)methyl)-2-octenediamide,
14) (E)-N1-cyclohexyl-N8-hydroxy-2-((naphthalen-1-yloxy)methyl)-2-octenediamide,
15) (E)-N8-hydroxy-2-((naphthalen-1-yloxy)methyl)-N1-(thiophen-2-ylmethyl)-2-octenediamide,
16) (E)-N8-hydroxy-N1-(4-methoxyphenethyl)-2-((naphthalen-1-yloxy)methyl)-2-octenediamide,
17) (E)-N8-hydroxy-2-((naphthalen-1-yloxy)methyl)-N1-(4-(trifluoromethoxy)benzyl)-2-octenediamide,
18) (E)-N1-(1-(cyclohexylmethyl)pyrrolidin-3-yl)-N8-hydroxy-2-((naphthalen-1-yloxy)methyl)-2-octenediamide,
19) (E)-N1-(1-cyclopentylpiperidin-4-yl)-N8-hydroxy-2-((naphthalen-1-yloxy)methyl)-2-octenediamide,
20) (E)-N1-(1-benzylpyrrolidin-3-yl)-N8-hydroxy-2-((naphthalen-1-yloxy)methyl)-2-octenediamide,
21) (E)-N8-hydroxy-N1-(1-isopropylpyrrolidin-3-yl)-2-((naphthalen-1-yloxy)methyl)-2-octenediamide,
22) (E)-N1-(1-(cyclohexanecarbonyl)pyrrolidin-3-yl)-N8-hydroxy-2-((naphthalen-1-yloxy)methyl)-2-octenediamide,
23) (E)-3-(8-(hydroxyamino)-2-((naphthalen-1-yloxy)methyl)-8-oxo-2-octenamido)pyrrolidine-1-carboxylic acid t-butyl ester,
24) (E)-N8-hydroxy-2-((naphthalen-1-yloxy)methyl)-N1-(pyrrolidin-3-yl)2-octenediamide,
25) (E)-N1-(1-cyclohexylpyrrolidin-3-yl)-N8-hydroxy-2-((naphthalen-2-yloxy)methyl)-2-octenediamide,
26) (E)-N1-(1-cyclopropylpyrrolidin-3-yl)-N8-hydroxy-2-((naphthalen-1-yloxy)methyl)-2-octenediamide,
27) (E)-N1-(1-cyclopropylpiperidin-4-yl)-N8-hydroxy-2-((naphthalen-1-yloxy)methyl)-2-octenediamide,
28) (E)-N1-(1-ethylpiperidin-4-yl)-N8-hydroxy-2-((naphthalen-1-yloxy)methyl)-2-octenediamide,
29) (E)-N1-(1-ethylpyrrolidin-3-yl)-N8-hydroxy-2-((naphthalen-1-yloxy)methyl)-2-octenediamide,
30) (E)-N8-hydroxy-N1-(2-(1-methylpyrrolidin-2-yl)ethyl)-2-((naphthalen-1-yloxy)methyl)-2-octenediamide,
31) (E)-N8-hydroxy-N1-(1-isopropylpiperidin-4-yl)-2-((naphthalen-1-yloxy)methyl)-2-octenediamide, and
32) (E)-N1-(3-(dimethylamino)propyl)-N8-hydroxy-2-((naphthalen-1-yloxy)methyl)-2-octenediamide.

3. The pharmaceutical composition according to claim 1, wherein the coating layer is present in an amount of 1 to 10% by weight based on weight of the tablet or capsule.

4. The pharmaceutical composition according to claim 1, wherein the composition is a pharmaceutical composition for injection having a liquid or lyophilized form.

5. A pharmaceutical composition for an anticancer agent comprising the compound of formula 1, the compound of formula 2, or a combination thereof according to claim 1.

6. A method for preparing a pharmaceutical composition comprising a compound of formula 1, a compound of formula 2 or a combination thereof, the method comprising:
1) adding an organic solvent to an alkylcarbamoyl naphthalenyloxy octenoyl hydroxyamide compound or a derivative thereof to extract a free base, and
2) adding phosphoric acid or tartaric acid to the solution of the free base,
wherein the pharmaceutical composition is in the form of a tablet, granule, powder, capsule, dry syrup or injection: 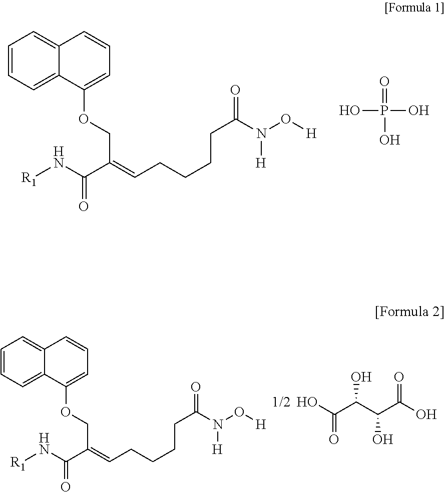
in the formula 1 or 2,
R₁ is C₁₋₃ alkyl unsubstituted or substituted with one or more substituents selected from the group consisting of halophenyl, C₁₋₃ alkoxy, C₁₋₃ alkoxy C₁₋₃ alkyl, cyclohexanyl, furanyl, thiophenyl, imidazole, imidazolidyl C₁₋₃ alkyl, C₁₋₃ alkylamino, di-C₁₋₃ alkylamino, hydroxyphenyl, tetrahydrofuranyl, cyclohexyl, cyclohexenyl, oxopyrrolidinyl, C₁₋₃ alkoxyphenyl, di-C₁₋₃ alkylaminophenyl, C₁₋₃ alkylpyrrolidinyl and trifluoromethoxyphenyl; pyrrolidine unsubstituted or substituted with C₃₋₈ cycloalkyl, C₃₋₈ cycloalkyl C₁₋₃ alkyl, benzyl, C₁₋₃ alkyl or C₃₋₈ cycloalkylcarbonyl; piperidine substituted with C₁₋₃ alkyl or C₃₋₈ cycloalkyl; furan; or C₃₋₈ cycloalkyl,
with proviso that unsubstituted C₁₋₂ alkyl and C₁₋₂ alkyl substituted with C₁₋₂ alkylpyrrolidinyl are excluded.

7. A method for preparing a pharmaceutical composition according to claim 6, further comprising additionally adding a solvent having a lower solubility than the organic solvent of step 1).

8. A method for preparing a pharmaceutical composition according to claim 6, wherein the organic solvent of step 1) comprises one or more selected from the group consisting of methanol, ethanol, propanol, tetrahydrofuran, chloroform, N,N-dimethylformamide (DMF), dimethyl sulfoxide (DMSO), acetonitrile and ethyl acetate.

9. A method for preparing a pharmaceutical composition according to claim 7, wherein the solvent having a lower solubility than the organic solvent of step 1) comprises one or more selected from the group consisting of alcohols including methanol, ethanol and propanol, tetrahydrofuran, acetonitrile and acetone.

10. A method for preparing a pharmaceutical composition according to claim 6, wherein the method further comprises sterilizing by a high-temperature decompression sterilization method or aseptic filtration method.
